(19) United States
(12) Patent Application Publication
(10) Pub. No.: US 2017/0043069 A1
(43) Pub. Date: Feb. 16, 2017

(54) NEGATIVE PRESSURE WOUND THERAPY REMOVAL DEVICE

(71) Applicant: Greenville Health System, Greenville, SC (US)

(72) Inventors: David L. Cull, Greenville, SC (US); John David Cull, Greenville, SC (US)

(21) Appl. No.: 15/220,471

(22) Filed: Jul. 27, 2016

Related U.S. Application Data
(60) Provisional application No. 62/203,085, filed on Aug. 10, 2015.

(51) Int. Cl. A61M 1/00; A61F 13/00; A61M 27/00

(52) U.S. Cl. CPC A61M 1/0088; A61M 1/0023; A61M 27/00; A61F 13/00068; A61M 1/0066; A61F 2013/00536

(57) ABSTRACT

Negative pressure wound therapy systems are described that can prevent the retainment of wound fill material in a wound cavity during dressing changes. The negative pressure wound therapy system includes a removal device that can be used to lift the wound fill material out of a wound cavity and prevent fracture of the wound fill material and retainment of fragments of the material in the wound. The removal device can include fibers (e.g., a fibrous net) that can be retained between the wall of the wound cavity and the wound fill material, with the wall of the wound and the wound fill material retaining direct contact with one another to encourage healing of the wound according to the therapy treatment process.

### CROSS REFERENCE TO RELATED APPLICATION

This application claims filing benefit of U.S. Provisional Patent Application Ser. No. 62/203,085 having a filing date of Aug. 10, 2015, which is incorporated herein by reference.

### BACKGROUND

Negative pressure wound therapy is often used in treatment of deep, burrowing, chronic wounds associated with diabetic ulcers, complex decubitus ulcers, and open abdominal wounds from laparotomy. It is commonly used to treat acute wounds in hospitalized patients and chronic wounds in patients being treated in an outpatient setting or in wound care centers. As illustrated in FIG. 1, the key elements of this therapy include an absorbent wound fill material 1, generally an open celled absorbent foam that fills the cavity of the wound 2, an air-tight dressing 3 on the skin surface that covers the wound fill material 1 and wound 2, and tubing 4 connected to a vacuum pump and fluid collection vessel. The use of negative pressure wound therapy in wound management has increased dramatically over the past 20 as it has been shown to promote wound healing.

Direct contact between the wound fill material 1 and the cavity wall of the wound 2 is essential to the successful use of negative pressure wound therapy. This contact allows fluids emanating from the wound surface to be removed through the wound fill material rather than collecting in dead spaces between the fill material and the wound. Also, through a mechanism that is poorly understood, the vacuum applied through the fill material stimulates healing at the material/wound interface. Unfortunately, this necessary contact can lead to fracture of the wound fill material and retainment of material fragments in the wound during dressing changes, which is a well-recognized complication of negative pressure wound therapy. Fragment retainment can result in infection that can exacerbate wound issues, slow healing, and lead to surgical intervention, even amputations or death in serious cases. It has been estimated that approximately 20% of surgeons in general and orthopedic surgery fields have either experienced such complications in their own patients or heard of it happening to the patient of a colleague.

Attempts have been made to prevent retainment of wound fill material through improved training. For instance, wound care nurses are specifically trained to recognize and prevent this complication, and caring for complex wounds is often the only responsibility of nursing professionals, with wound care often being carried out by teams of nurses to further prevent complications. Unfortunately, accidental oversight of wound fill material fragments can still take place during wound treatment. Moreover, the high level of care found in hospital settings is not possible in the outpatient setting. Outpatient nurses do not focus on only wound care and do not typically change dressings in teams. Moreover, the nurse providing care to the patient in the outpatient setting may change week to week. This situation can lead to retainment of a small piece of the wound fill material in a burrowing wound, and granulation tissue may eventually cover the piece of material, sealing it off from the rest of the wound.

There is a need in the art for devices to aid in complete removal of wound fill materials for use in negative pressure wound therapy protocols. Moreover, devices that can aid in removal of wound fill material without interfering with the beneficial interaction between the wound cavity wall and the wound fill material during a treatment protocol would be of great benefit.

### SUMMARY

According to one embodiment, disclosed is a negative pressure wound treatment system that includes a wound fill material and a removal device. The removal device is configured to be held in conjunction with the wound fill material such that during use a first portion of the removal device is located between the wound fill material and a wall of a wound cavity and a second portion of the removal device extends from the wound. More specifically, the removal device is configured to be held in conjunction with the wound fill material such that the first portion of the removal device will contact about 10% or less of the external surface area of the wound fill material thus allowing for a high degree of direct contact between the wound fill material and the wall of the wound cavity.

Also disclosed is a method for treating a wound that includes locating a removal device within a wound cavity such that a first portion of the removal device is adjacent to a wall of the wound cavity and a second portion of the removal device extends out of the wound. The method also includes locating a wound fill material in the wound cavity such that the first portion of the removal device is between the wound fill material and the wound cavity wall and such that the first portion of the removal device is in contact with about 10% or less of the external surface area of the wound fill material and about 50% or more of the external surface area of the wound fill material is in direct contact with the wall of the wound cavity.

### BRIEF DESCRIPTION OF THE FIGURES

The present disclosure may be better understood with reference to the following figures:

FIG. 1 illustrates a prior art example of a negative pressure wound therapy treatment system.

FIG. 2 illustrates one embodiment of a wound treatment system including a wound fill material and a removal device.

FIG. 3 illustrates another embodiment of a removal device.

FIG. 4 illustrates another embodiment of a removal device.

FIG. 5 illustrates another embodiment of a removal device.

FIG. 6 illustrates another embodiment of a removal device.

FIG. 7 illustrates another embodiment of a removal device.

FIG. 8 illustrates another embodiment of a removal device.

FIG. 9 illustrates another embodiment of a removal device.

FIG. 10 illustrates a negative pressure wound treatment system including a removal device.

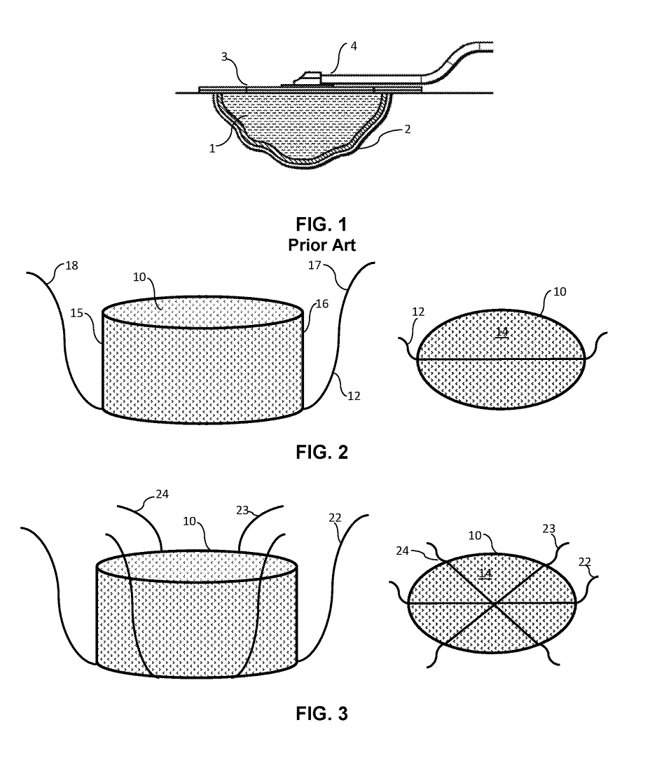

### DETAILED DESCRIPTION

It is to be understood by one of ordinary skill in the art that the present discussion is a description of exemplary embodiments only, and is not intended as limiting the broader aspects of the present invention.

The present disclosure is generally directed to negative pressure wound therapy systems that can prevent the retainment of wound fill material in a wound cavity during dressing changes. More specifically, the negative pressure wound therapy system includes a removal device that can be used to lift the wound fill material out of a wound cavity and prevent fracture of the wound fill material and retainment of fragments of the material in the wound.

Negative pressure wound therapy fill material is designed to not fracture, even in areas where tissue ingrowth has occurred, if it is lifted off the broad surface area of the wound. However, in a burrowing wound, more than 90% of the external surface area of the wound fill material can be in contact with the wound surface. To remove the fill material from the wound cavity, a pulling force is applied to the small portion of the fill material that is exposed at the wound surface. The forces required to remove the fill material in these situations can exceed the material strength, particularly if tissue in-growth in the fill material has occurred, resulting in fill material fracture and the formation of fragments that can be missed by the practitioner when cleaning the wound. The removal devices described herein include a first portion that is configured to be interposed between the wound fill material and the surface of the open wound. The removal devices also include a second portion that extends from the wound. During use, force can be applied to the second portion. This force can then be transferred to the first portion as a lifting force to remove the wound fill material from the wound cavity. Because the removal forces are applied as a lifting force to the lower surface and/or interior of the fill material within the wound cavity, rather than as a pulling force to the exposed material at the wound surface, the wound fill material can be completely removed from the wound cavity with less likelihood of fracture and less likelihood of retention of fill material in the wound. This can provide an added degree of safety to patients being treated with negative pressure wound therapy.

The removal device includes a portion that during use can be located between the wound fill material and the wall of the wound cavity without interfering with the interaction between the wound fill material and the wound cavity wall. For instance, following insertion of a removal device and a wound fill material in a wound cavity, about 50% or more, about 60%, about 70% or more, about 80% or more, or about 90% or more in some embodiments, of the external surface of the wound fill material (i.e., excluding the surface area of any internal porosity of a material) can be in direct contact with the wall of a wound cavity.

To prevent excessive interference in contact between the wound fill material and the wound cavity wall, at least that portion of the removal device that is configured for location within a wound cavity can be formed of one or more components having a relatively small cross sectional area. For instance, a removal device can be formed of one or more tapes, ribbons, fibers or the like having a relatively small cross sectional dimension, e.g., about 1 millimeter or less in a maximum cross sectional dimension. In one embodiment, a removal device can include one or more fibers, for instance one or more fibers having a size equivalent to a suture of from about a #5 suture to about a #11-0 suture. The small diameter structures can contact little of the exterior surface of the wound fill material. For instance, during use, the portion of the removal device held in contact with the wound fill material can contact about 10% or less, about 5% or less, about 2% or less, or about 1% or less of the external surface area of the wound fill material, leaving a high surface area free for direct contact with the wall of the wound cavity.

The removal device can be formed of a biologically inert material that can be safely located within a wound for a period of time. By way of example, a removal device can include a polymeric composition of one or more polymers. Polymers for use in forming a removal device can include non-absorbable polymers such as, without limitation, polyamide (e.g., nylon), polyester (e.g., polyethylene terephthalate such as Dacron®), polyvinylidene fluoride (PVDF), polyolefins (e.g., polypropylene), etc., or mixtures thereof. In some embodiments, the removal device can include one or more absorbable polymers such as, without limitation, polyglycolic acid, polylactic acid, glycolide/ε-caprolactone copolymer (e.g., Monocryl®), polydioxanone, or mixtures of polymers. A removal device can include one or more elastomeric polymers, e.g., polyurethane elastomers and the like, optionally in conjunction with one or more additional polymers that can provide a removal device with elastic characteristics. Elastic characteristics can be useful to improve conformation of the removal device with the wound cavity following location of the device between the wound cavity surface and the wound fill material.

Fibers, tapes, ribbons, etc. as may be utilized in forming a removal device can include yarns, multi-filament fibers, monofilament fibers, or some combination thereof. Multi-filament fibers or yarns can generally include between about 5 and about 100 individual filaments of the same or different materials, usually including some twist in the yarn. Moreover, fibrous materials as may be encompassed in a removal device can include multi-component fibers including core/sheath fibers, islands-in-the-sea fibers, braided fibers, and so on, as well as fibers including adjacent lengths of different materials. A removal device can include structures of different materials in any combination, for example a non-absorbable filament in combination with an absorbable filament. Additionally, a removal device can be fabricated using different types of fibrous materials in combination, for example, a yarn in connection with a suture.

Any suture material as is known in the art can be utilized. Suture material for an implantable device can be absorbable or non-absorbable, as desired. Suture can be of any size (e.g., from #11-0 up to #5 in size). Suture can be multifilament and braided or twisted, or can be mono-filament. Suture can be sterile or non-sterile, of natural, synthetic, or a combination of materials.

In one embodiment, the fibrous material of the removal device can be coated. Typical coatings can include, for example, collagen, magnesium stearate, PTFE, silicone, polybutilate, and antimicrobial substances.

A large variety of suitable suture is known to those of skill in the art and can include, without limitation, collagen, catgut, polyglycolic acid, polyglactin 910 (e.g., Vicryl®), poliglecaprone 25 (e.g., Monocryl®), polydioxanone, surgical silk, surgical cotton, nylon, polybutester, polyester, polyethylene, polypropylene (e.g., Prolene®), and the like. For instance, polyethylene suture such as co-braided polyethylene suture can be utilized in the removal device.

The negative wound pressure system utilizes other typical components of a system in conjunction with the removal device including, without limitation, a wound fill material, an air-tight dressing, suction pumps, tubing, drainage reservoirs, etc., embodiments of which are discussed further herein.

The wound fill material can be any suitable wound fill material, with the preferred material generally depending upon the type of wound and the specific characteristics of the treatment to be carried out. In general, the wound fill material can be a polymeric foam, e.g., a polyurethane foam, a polyester foam, a polyether foam or a polyvinyl alcohol foam. The wound fill material can be hydrophobic or hydrophilic, depending upon the nature of the wound and the treatment. For instance, a polymeric foam can be hydrophilic as formed or can be a hydrophobic foam treated with an agent that makes the foam hydrophilic.

Highly absorbent foams as are known in the art can be utilized as a wound fill material. By way of example, U.S. Pat. No. 8,772,567 to Eckstein, et al., which is incorporated herein by reference, describes a polyurethane foam for use in negative pressure wound therapy as may be utilized.

GranuFoam™, marketed by Kinetic Concepts Inc., San Antonio, Tex. is a black polyurethane foam that can be utilized in conjunction with a removal device. Black foam is preferably utilized in negative pressure wound therapy in one embodiment as the foam is easily identifiable from other foams that may be utilized in the wound care treatment. A drawback of using black foam has been that a user or a care taker may not be able to clearly see signs of infection on the foam during dressing changes, and the black color of the foam may make it difficult to see fragments retained in the wound during removal. Black foam is generally a polyurethane foam with a hydrophobic, reticulated open-cell structure (400 to 600 μm diameter pores). Black foam can provide a more uniform distribution of negative pressure and is used most commonly in highly exudating wounds and deep cavity wounds.

WhiteFoam™ also marketed by Kinetic Concepts Inc, San Antonio, Tex. is a white polyvinyl alcohol based foam with a hydrophilic (saline moistened), non-reticulated, higher-density cell structure (200 to 1000 μm porosity). A white foam can be preferred in some embodiments as it can prevent granulating skin tissue from growing into the dressing and can be easier to handle when placing in and removing from tunnels and small cavities because of its higher tensile strength. A disadvantage of the white foam is that it doesn't provide uniform suction across the material and therefore doesn't remove exudate from the wound as effectively as the black foam. While a white foam may be initially more clearly distinguishable within the wound cavity, in practice, following a period of time in the wound a white foam will become discolored and no longer clearly distinguishable. Thus, use of a white foam alone also carries risk of fragment retainment in a wound. Beneficially, the removal device can be utilized with any color or type of wound fill material.

While wound fill material for use in a negative pressure wound therapy protocol is generally designed for use as a single piece of material that is trimmed as necessary to snugly fig into a wound cavity, in practice, this is not always the case. Occasionally, and particularly in those cases in which a dressing is changed by one not specifically trained in wound care treatment, the wound fill material can be utilized as a plurality of separated pieces of material, rather than as a single unit. The removal device can be of great benefit in such embodiments, as the lifting action of the device can remove all of the wound fill material from the wound cavity, even in those cases in which the wound fill material has been packed into the wound in multiple segments that can be quite difficult to see.

The removal device is designed to be utilized in conjunction with a wound fill material to allow maximum direct contact between the wound surface and the wound fill material. As previously stated, a removal device can include a single component such as a fiber or a plurality of components (e.g., a plurality of fibers) combined together. FIG. 2 illustrates one embodiment of a system including a wound fill material 10 and a removal device 12. It should be understood that while the wound fill material is illustrated as a generally cylindrical segment of material, during use the wound fill material can be trimmed as necessary to snugly fit into a wound cavity. Thus, any shape of wound fill material and any number of separate pieces of wound fill material can be utilized with the removal system.

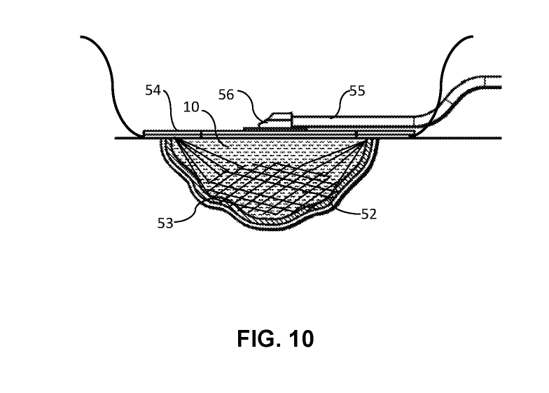

In the embodiment of FIG. 2, the removal device 12 includes a single fiber that during use is located in a wound cavity so as to pass beneath the wound fill material 10, as shown in a front view (left) and a bottom view (right) of the system. The removal device 12 is of a length to extend along the lower surface 14 of the wound fill material 10 and along the sides 15, 16 of the wound fill material 10 as shown. In addition, the removal device includes portions 17, 18 that during use can extend out of the wound (see, e.g., FIG. 10).

The removal device 12 can be attached to the wound fill material 10 or can be merely held adjacent to the wound fill material during use, as desired. For instance, the removal device 12 can be melt bonded to the wound fill material in those embodiments in which the two components are formed of suitable materials for melt bonding to one another. Alternatively, a suitable bioadhesive can be utilized to adhere the removal device 12 to the wound fill material at desired locations. For example, the single fiber removal device 12 of FIG. 2 can be adhered to the wound fill material along the bottom surface 14 of the wound fill material so as to properly maintain the alignment of the two following placement in a wound cavity. Other attachment mechanisms such as, without limitation, knotting, hooks, clips, and so forth can be utilized to attach the wound fill material 10 to a removal device 12.

Of course, a removal device is not limited to a single fiber as illustrated in FIG. 2. By way of example, in the embodiment illustrated in FIG. 3, the removal device includes a plurality of fibers 22, 23, 24, that together are located in conjunction with a wound fill material 10 during packing of a wound with the wound fill material 10. The system is illustrated in a front view (left) and a bottom view (right) in FIG. 3 to illustrate one possible arrangement of the fibers 22, 23, 24 when held in conjunction with the wound fill material 10. As shown, the fibers 22, 23, 24 can all pass across the lower surface 14 of the wound fill material 10; the lower surface 14 being that portion of the wound fill material 10 that will be at the base of a wound cavity during use.

The multiple fibers 22, 23, 24 can be adhered to one another and/or to the wound fill material 10 as desired. In addition, it should be understood that utilization of a plurality of fibers is not limited to three fibers as illustrated, and any number of fibers and any arrangement of the fibers is encompassed herein. For instance, in those embodiments in which the fibers are attached to one another, not all of the fibers need include a portion that extends beyond the wound fill material to be available exterior to the wound for removal of the wound fill material from the wound cavity. In addition, the fibers 22, 23, 24 can pass around a portion of the wound fill material according to any desired geometry, i.e., they need not all contact one another at a central point of the lower surface 14 as shown in FIG. 3. In fact, a plurality of fibers need not contact one another within the wound cavity at all, and they can each pass around a different portion of the wound fill material independently of one another in some embodiments.

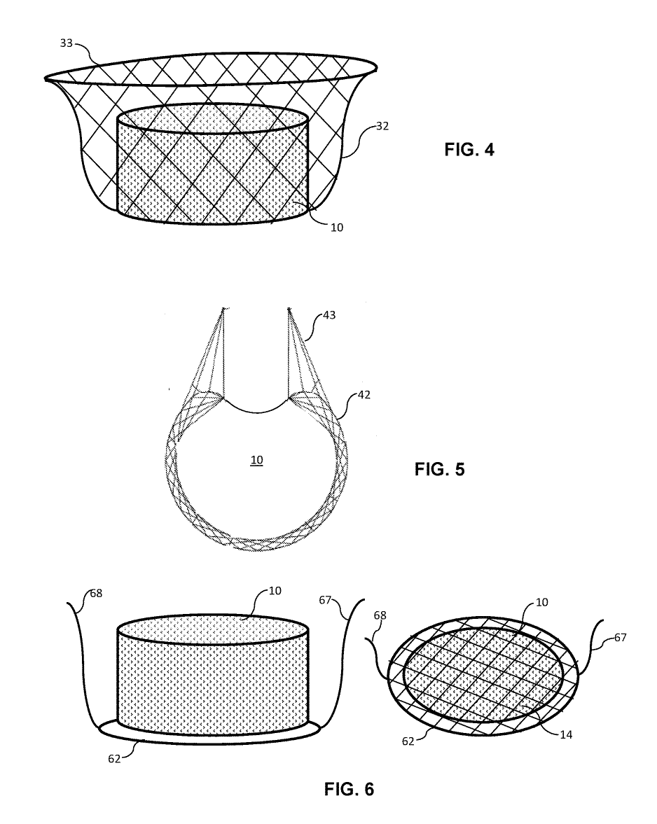

FIG. 4 illustrates an embodiment of a removal device in the form of a fibrous retrieval net 32. The interstices between the individual fibers of the retrieval net 32 are sufficiently wide so as to allow for the desired contact between the wound fill material 10 and the wall of a wound cavity. In addition, the interstices are large enough to allow the retrieval net to conform to the surface of wounds of various shapes and sizes with minimal in-folding. The interstices are sufficiently wide so that even in areas where in-folding may occur, the wound fill material 10 remains in contact with a maximal amount of the wound surface. Accordingly, the retrieval net 32 can generally have interstices with a minimum cross sectional dimension of about 0.25 inches (about 0.65 millimeters) or greater, for instance about 0.4 inches (about 10 millimeters) or greater, or about 0.5 inches (about 12 millimeters) or greater in some embodiments.

As illustrated in FIG. 4, the retrieval net 32 can be large enough to be inserted in a wound cavity between the wall of the wound cavity and the wound fill material 10 with a portion 33 of the retrieval net 32 extending beyond the wound fill material 10 and out of the wound. Thus, to remove the wound fill material from the wound, the portion 33 of the retrieval net 32 that extends out of the wound can be simply pulled to lift the wound fill material 10 and the retrieval net 32 out of the wound cavity.

According to one embodiment, a large flat webbing material can be utilized to form a retrieval net 32. For instance, a webbing material can be provided that is wrapped around a dowel. During use, a portion of the webbing material can be unrolled from the dowel, trimmed to the appropriate size and shape and placed over/in a wound cavity. The wound fill material 10 can then be trimmed as necessary and packed into the wound against all wound surfaces on top of the retrieval net 32. The edges of the retrieval net 32 can extend beyond the most superficial edge of the wound fill material 10 as shown in FIG. 4. To change the wound fill material, the portion 33 of the retrieval net 32 that extends beyond the wound cavity can be grasped and pulled to facilitate and ensure complete removal of the wound fill material 10 from the wound.

A retrieval net need not fill the wound cavity. For instance, in the embodiment illustrated in FIG. 5, a retrieval net 42 is designed to pass around a portion of the wound fill material 10, including the lower surface 14 of the wound fill material 10 that will be at the bottom of a wound cavity during use in the fashion of a sling. In this embodiment, a larger portion of the wound fill material 10 can be in direct contact with the wall of the wound cavity with no portion of the retrieval net 42 between the two. As shown, in this embodiment, the portion 43 of the removal device that extends beyond the wound cavity includes fibrous extensions of portions of the net 42, rather than the complete webbing as in the embodiment of FIG. 4.

As with a removal device formed of individual fibers, a retrieval net can be pre-attached the wound fill material at a single or several locations. For instance, the retrieval net 42 of FIG. 5 can be attached to the wound fill material 10 at the lower surface 14 of the wound fill material that is intended to be located at the base of a wound cavity. During use, the wound fill material 10 and the retrieval net 42 can be trimmed to match the wound size and shape.

The portion of the removal device that extends from the wound cavity and the portion that is located between the wall of the wound cavity and the wound fill material need not be extensions of the same structure. For instance, the embodiment illustrated in FIG. 6 includes a retrieval net 62 that can be located adjacent to the wound fill material 10 such that it passes over at least the lower surface 14 of the wound fill material 10 that will be located at the base of a wound cavity. Removal device also includes portions 67, 68 that extend from the retrieval net 62. These portions 67, 68 can be long enough to extend out of the wound cavity during use. Portions 67, 68 can be of unitary construction with the retrieval net 62 or can be separate materials that can be affixed to the retrieval net 62 during use. For example, in one embodiment, prior to placing the wound fill material 10 in the wound cavity, portions 67, 68 can be affixed to the retrieval net 62 that will be placed at the deepest portion of the wound. Portions 67, 68 can be affixed to the retrieval net 62 according to any fashion including use of a bioadhesive, knotting, clips, hooks, etc.

The portions 67, 68 can extend from the retrieval net 62, between any remaining portion of the wound cavity and the wound fill material 10, and beyond the wound surface. To remove the wound fill material, the portions 67, 68 can be pulled to dislodge the deepest portion 14 of the wound fill material 10 from the wound surface. Once dislodged, the wound fill material 10, the retrieval net 62, and the portions 67, 68 can be lifted out of the wound as a unit.

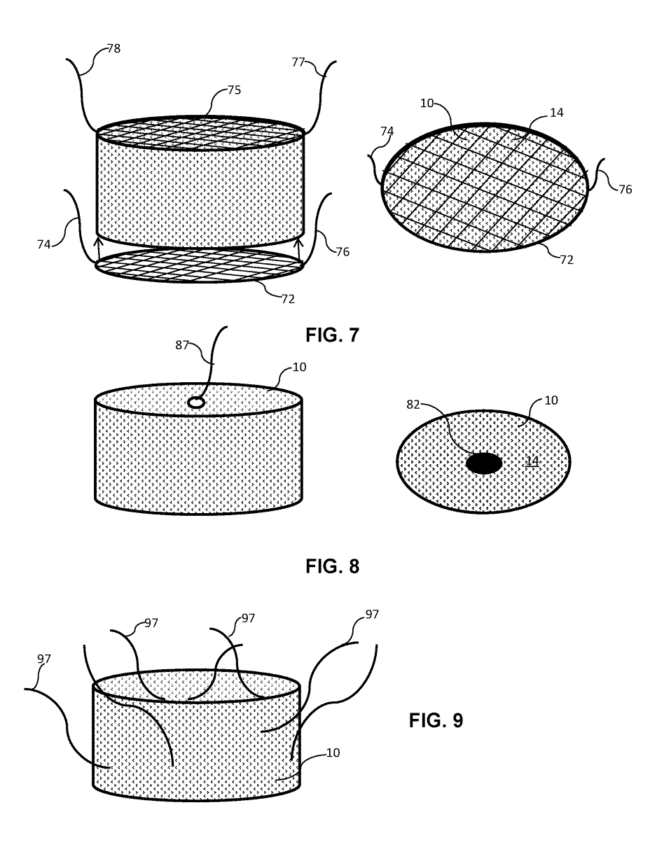

According to the embodiment illustrated in FIG. 7, a wound fill material 10 can include a first removal device portion 72 and a second removal device portion 75. In this particular embodiment, the portions 72 are in the form of open weave netting, but this is not a requirement and in other embodiments, the portions 72, 75 can be formed of one or more fibers, tapes, ribbons, etc. in any suitable orientation, either connected to one another or separate, as desired. As shown, the portions 72 75 can be applied to opposite sides of the wound fill material 10. For instance, the portions 72, 75 can be bonded, sewn, or otherwise attached to the wound fill material 10. During use, the wound fill material 10 including the portions 72, 75 on either side can be trimmed to fit the size and shape of a wound with one of the portions 72, 75 to be located at the base of the wound cavity. The system can also include several fibrous lines 74, 76, 77, 78 one or more of which can be attached to at least that portion 72 or 75 that will be at the base of the wound cavity. Optionally, one or more of the lines 74, 76, 77, 78 can also be attached to that portion 72 or 75 that is on the opposite side of the wound fill material, to help during removal of the system, but this is not a requirement of the removal system. The fibrous lines can be tied to the portions 72, 75, or can be attached by any suitable device including, without limitation, hooks, clips, adhesives, melt bonding, etc. Of course, any number of lines can be attached to the bottom and the top portions 72, 75 for removal of the wound fill material 10, and the device is not limited in any way to the illustrated number. The bottom view (right) of the wound fill material 10 in FIG. 7 illustrates the portion 72 covering the lower surface 14 of the wound fill material 10 with the large interstices of the portion 72 allowing contact between the wound fill material and the wall of the wound cavity during use.

A removal device is not limited to fibrous components. For instance, in one embodiment illustrated in FIG. 8 a removal device can include a disc 82 that during use can have a line 87 attached thereto. According to this embodiment, prior to packing the wound fill material 10 into the wound, the line 87 that is attached to the disc 82 can be passed through the wound fill material 10 from a lower surface 14 where the wound fill material will contact the deepest portion of the wound. For instance, a needle can be used to pass the line 87 through the wound fill material from a lower surface 14 and out of an opposite side of the wound fill material. Following the passage of the line 87 through the wound fill material, the needle can be detached from the line 87 and the line 87 can be pulled, thereby securing the disc 82 against the lower surface 14 of the wound fill material 10. A disc can be, for example a molded polymeric disc formed of polymers as described previously or a biocompatible metal disc, e.g., nitinol, titanium, silver, etc.

During use, the wound fill material 10 can be packed into the wound with the disc 82 at the base of the wound. At some later time, in order to remove the wound fill material 10, the line 87 that passes through the wound fill material and extends out of the wound can be pulled to lift and dislodge the wound fill material 10 from the wound base. The wound fill material 10 and the removal device including the disc 82 and the fibrous line 87 can then be removed from the wound as a unit.

FIG. 9 illustrates yet another embodiment of a removal device in which the wound fill material 10 has been formed to include a plurality of fibers 97 throughout the wound fill material. The plurality of fibers 97 can be randomly located throughout the wound fill material 10 or can be provided as a woven mat or in some other organized fashion. In any case, the fibers 97 can extend from the wound fill material such that upon packing the wound fill material 10 into a wound cavity at least a portion of the fibers 97 can extend from the wound. This embodiment can also prevent fracturing of the sponge upon its removal.

Multiple removal devices can be utilized together, as desired. For example, a removal device as in FIG. 9 that includes a plurality of fibers throughout a wound fill material may be beneficially utilized in conjunction with a retrieval net as described in other embodiments. Any combination of removal devices may be utilized to improve removal of wound fill material from a wound cavity.

A removal device can include additional components as may be desired. For instance, in one embodiment a removal device can include a radiopaque marker that can be affixed to the device as a safety feature. In the event the removal device were used inappropriately (e.g., the edge of a retrieval net were left in the wound, or a line attached to a retrieval net were to become detached), a plain radiograph of the wound could be obtained to visualize the retained portion.

A negative wound therapy system in accordance with the present disclosure can include other standard components as are generally known in the art. For example, and with reference to FIG. 10, in addition to a wound fill material 10 and a removal device 52, a system can include an air-tight cover material 54 for sealing of the wound space 53. The wound space 53 being generally regarded as the wound cavity and the area immediately surrounding the wound. In accord with standard practice in the art, “air-tight sealing” does not mean that there is no exchange of gas between the wound space and its surroundings. Rather, air-tight sealing in this context means that, taking into account the vacuum pump used, the negative pressure necessary for the negative pressure wound therapy can be maintained. This means that cover materials can be used which have a slight degree of gas permeability as long as the negative pressure necessary for the negative pressure wound therapy can be maintained.

The cover material 54 can be fastened in the area of the wound space 53 and sealed according to standard practice. This can be achieved, for example, by providing an adhesive edge on the cover material 54. Alternatively, an adhesive substance can be applied either to the edge of the cover material 54 and/or the intact skin around the wound space 53. This approach has the advantage that it is easier to match the cover material to the shape and size of the wound.

In one embodiment, the cover material 54 can include a water-insoluble polymer and/or a metal foil. For instance, a water-insoluble polymeric cover material can have a solubility of 10 mg/L or less, or 1 mg/mL or less, or from about 0.0001 mg/mL to about 1 mg/mL in some embodiments. Solubility can be determined in accordance with the column elution method pursuant to EU Directive RL67-548EEC, annex V, chapter A6. A cover material 54 can be formed of polymers such as, and without limitation, polyurethane, polyester, polypropylene, polyethylene, polyamide, polyvinyl chloride, polyorganosiloxane (silicone), or a mixture thereof.

A system can also include a connection, e.g., silicon tubing 55 to connect the wound space 53 to a negative pressure source (not illustrated in FIG. 10). The tubing 55 can generally include a connection 56 that is external to the cover material 54 to generate a negative pressure in the wound space 53 and allow fluids to be removed from the wound space 53 via the tubing 55. The connection 56 can include ducting of the tubing 55 through the cover material 54. Alternatively, the tubing 55 can be led under an edge of the cover material 54. In any case the connection 56 can be sealed air-tight so that the desired negative pressure can be maintained in the dressing.

In one embodiment, the connection 56 can include a negative pressure connector (port) that can be fastened to one of the inner or outer sides of the cover material 54, whereby the cover material 54 has the corresponding openings. In this embodiment it is also important to ensure air-tight sealing either of the penetration opening (inside port) or the surface of the dressing (outside port). Sealing can be achieved, for example, with an adhesive foil, an adhesive paste or an adhesive strip.

The expression “negative pressure in the wound space” as utilized herein generally describes an air pressure that is lower inside the wound cavity compared to the atmospheric pressure. In one embodiment, the pressure difference between the atmospheric pressure and the pressure in the wound cavity during treatment can be from about 25 mm Hg to about 250 mm Hg, for instance from about 50 mm Hg to about 150 mm Hg, from about 80 mm Hg to about 140 mm Hg, or from about 120 mm Hg to about 130 mm Hg in some embodiments.

While certain representative embodiments and details have been shown for the purpose of illustrating the subject invention, it will be apparent to those skilled in this art that various changes and modifications may be made therein without departing from the scope of the subject invention.

What is claimed is:

1. A wound treatment system comprising:
a wound fill material, the wound fill material comprising an external surface area;
a removal device, the removal device being configured to be held in conjunction with the wound fill material such that during use a first portion of the removal device is located between the wound fill material and a wall of a wound cavity with the first portion of the removal device in contact with about 10% or less of the external surface area of the wound fill material, the removal device including a second portion that extends from the wound fill material when the first portion is held in contact with the wound fill material.

2. The wound treatment system of claim 1, wherein the removal device comprises a fiber or a fibrous net.

3. The wound treatment system of claim 2, the removal device comprising a fibrous net comprising interstices having a smallest cross sectional dimension of about 0.25 inches or greater.

4. The wound treatment system of claim 1, wherein the wound fill material and the removal device are permanently or removably attached to one another.

5. The wound treatment system of claim 1, the second portion of the removal device including a fibrous line.

6. The wound treatment system of claim 5, wherein the fibrous line is removably attachable to the first portion of the removal device.

7. The wound treatment system of claim 1, the removal device comprising a disc and a fibrous line, the first portion of the removal device including the disc and the second portion of the removal device including the fibrous line.

8. The wound treatment system of claim 7, wherein the disc and the fibrous line are permanently or removably attached to one another.

9. The wound treatment system of claim 1, the wound fill material comprising a foam.

10. The wound treatment system of claim 9, the foam comprising a polyurethane or a polyvinyl alcohol.

11. The wound treatment system of claim 1, further comprising a vacuum pump.

12. A method for treating a wound comprising:
locating a removal device in a wound such that a first portion of the removal device is adjacent to a wall of the wound and a second portion of the removal device extends out of the wound;
locating a wound fill material in the wound such that the first portion of the removal device is between the wound fill material and the wall of the wound and such that about 50% or more of an external surface area of the wound fill material is in direct contact with the wall of the wound and about 10% or less of the external surface area of the wound fill material is in contact with the first portion of the removal device.

13. The method of claim 12, further comprising applying suction to the wound fill material.

14. The method of claim 12, further comprising applying a cover material over the wound.

15. The method of claim 12, further comprising shaping the removal device to conform to the shape of the wound.

16. The method of claim 12, further comprising shaping the wound fill material to conform to the shape of the wound.

17. The method of claim 12, further comprising removing the wound fill material from the wound by use of the removal device.

18. The method of claim 12, further comprising attaching the wound fill material to the removal device.

19. The method of claim 12, the removal device comprising a fibrous line, the method further comprising attaching the fibrous line to a portion of the removal device.

20. The method of claim 12, wherein the wound is an acute wound, a surgical wound, or a chronic wound.
